(19) United States
(12) Patent Application Publication
(10) Pub. No.: US 2015/0289748 A1
(43) Pub. Date: Oct. 15, 2015

(54) MEDICAL ENDOSCOPE

(71) Applicant: OLYMPUS WINTER & IBE GMBH, Hamburg (DE)

(72) Inventor: Patrick Scherr, Moelln (DE)

(73) Assignee: OLYMPUS WINTER & IBE GMBH, Hamburg (DE)

(21) Appl. No.: 14/438,799

(22) PCT Filed: Oct. 22, 2013
(86) PCT No.: PCT/EP2013/003171

§ 371 Date: Apr. 27, 2015

(30) Foreign Application Priority Data
Nov. 6, 2012 (DE) 10 2012 021 614.9

(51) Int. Cl. A61B 1/00

(52) U.S. Cl. CPC A61B 1/00078; A61B 1/00167

(57) ABSTRACT

A medical endoscope including: a plurality of tubes arranged one inside the other; wherein an outer tube of the plurality of tubes is made of metal, and at least one inner tube of the plurality of tubes is made of ceramic, such as tungsten carbide. The at least one inner tube can be configured as a fiber tube inwardly delimiting a fiber chamber filled with optical fibers, where the fiber tube is made of ceramic, such as a sintered ceramic.

### CROSS-REFERENCE TO RELATED APPLICATION

The present application is based upon and claims the benefit of priority from PCT/EP2013/003171 filed on Oct. 22, 2013, which claims benefit to DE 10 2012 021 614.9 filed on Nov. 6, 2012, the entire contents of each of which are incorporated herein by reference.

### BACKGROUND

1. Field

The present invention generally relates to endoscopes, and more particularly to a medical endoscope of the type referred to in claim 1.

2. Prior Art

A plurality of tubes arranged one inside the other arises, e.g. from the construction described in DE 10 2004 023 866 B3, FIG. 1, in which the outermost tube is the fiber tube of an endoscope, wherein optical fibers are arranged between this fiber tube and a cladding tube surrounding the same. There thus arises a three-tube construction having, when viewed from outside inward, the outer tube, the fiber tube, and an inner, rotatably mounted tube for the rotary drive of the image sensor.

In the known construction, all tubes are designed as metal tubes. They provide the endoscope with substantial stiffness and protection against destruction caused by too much flexing.

The ongoing refinements of the surgical technicians demand increasingly thinner and longer endoscopes, which are subjected to ever increasing levels of bending. The strengths of the high-grade steels suitable for these intended uses are no longer sufficient.

Ceramics, e.g. tungsten carbide, can have sufficiently high flexural strengths. However, they form spall fractures when overloaded. Splinters are absolutely not acceptable during use of an instrument in a human body.

DE 10 2004 020 383 A1 describes an endoscope made of a metal-ceramic composite material, which combines high flexural strength with sufficient ductility, thus lower risk of breakage. The available material is tungsten carbide-cobalt. However, this material is controversial in medical technology because of possible risks to health.

### SUMMARY

An object of the present invention consists in creating a medical endoscope of the type listed at the beginning having a high degree of patient safety, by means of which increased bending forces can be achieved.

According to the invention, in a multiple-tube endoscope construction, one of the inner tubes is formed from unbreakable ceramic. As the outer tube consists of metal, the risk of splinters from an inner ceramic tube is not an issue. The potentially existing ceramic splinters cannot escape to the outside due to the outer metal tube. Patient safety is thus ensured. Also, the inner tube of a multiple-tube construction can provide the same with increased strength. There is also naturally the possibility of reducing the wall thicknesses at the same strength.

By this means, there arises for the first time the possibility of maintaining high patient safety and usable strength values, yet still build thinner and longer endoscopes.

According to claim 2, tungsten carbide is preferably used as the ceramic, which is distinguished by particularly high strength values.

With regard to the conventional construction of a multiple-tube endoscope, the outer tube serves as a cladding tube for outer covering and sealing. Between the outer tube and the second tube, the tube following the outer tube when viewed from outside inward, a fiber chamber is provided designed conventionally as crescent shaped in cross section, which chamber receives the optical fibers. The second tube inwardly delimits this chamber as the fiber tube. According to claim 3, the fiber tube is advantageously formed from ceramic. The tube with the particularly high strength thereby lies as far outward as possible, namely as the second outermost tube, by which means particularly high strength values result.

According to claim 4, the fiber tube is preferably formed as a sintered component. Ceramics are especially suited to sintering and thus allow the simple production of geometrically highly complex shapes, as are present in particular with regard to fiber tubes in the distal end regions thereof, as e.g. U.S. Pat. No. 5,718,664 shows. These complex contourings in the distal end regions of a fiber tube are particularly necessary for the correct alignment of the optical fibers in this region.

### BRIEF DESCRIPTION OF THE DRAWINGS

The invention is represented by means of example and schematically in the drawing. As shown in:

FIG. 1 illustrates a side view of a medical endoscope according to the invention,

FIG. 2 illustrates a section according to line 2-2 in FIG. 1, and

FIG. 3 illustrates a side view of a fiber tube contained in the endoscope.

### DETAILED DESCRIPTION

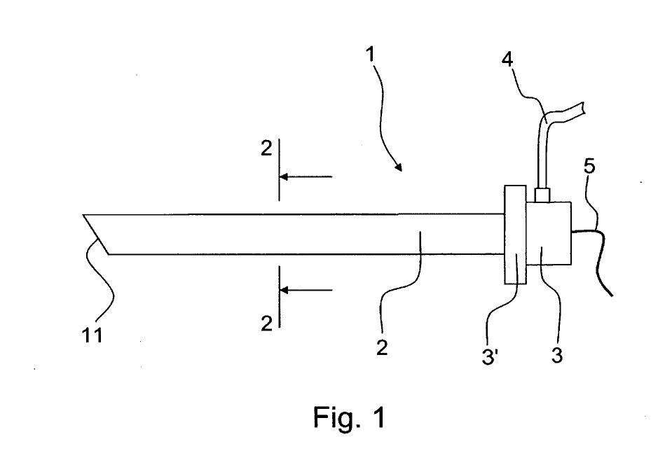

FIG. 1 shows the side view of a medical endoscope 1 comprising a shaft which is enclosed outwardly by a cladding tube 2 forming the outer tube. An end body 3, from which an optical fiber connection cable 4 and a video cable 5 branch off, sits on the proximal end of the cladding tube 2.

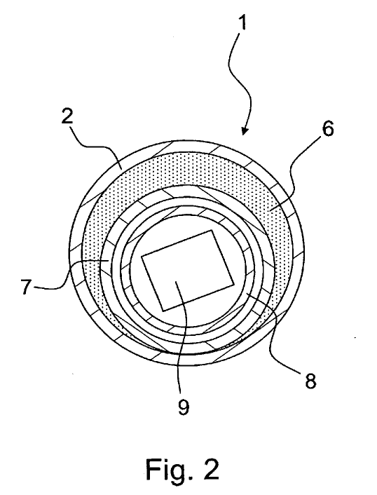

FIG. 2 shows a cross section according to line 2-2 in FIG. 1 with respect to a typical embodiment of a video endoscope, as described e.g. in DE 10 2004 023 866 B3, FIG. 1.

Cladding tube 2 encloses a fiber chamber 6, which contains optical fibers arranged in the direction of the extension of outer tube 2, which fibers are connected on the proximal ends thereof to the optical fiber connection cable 4. Fiber chamber 6 is delimited externally by cladding tube 2 and internally by a fiber tube 7, which forms, when considered from outside to inside, the second tube in the multiple-tube construction represented in FIG. 2.

In the inside of fiber tube 7, a system tube 8 of a video system is arranged, in the distal end region thereof an image sensor 9 is arranged, which is connected to video cable 5 via conductors (not shown). System tube 8 is rotatably mounted with respect to fiber tube 7, and can be adjusted into any angular position, according to the reasons that are explained in DE 10 2004 023 866 B3. For this purpose, a ring 3′ is rotatably mounted on end region 3, which ring rotatably drives the rotatably mounted system tube 8 using a suitable, e.g. magnetic rotational connection.

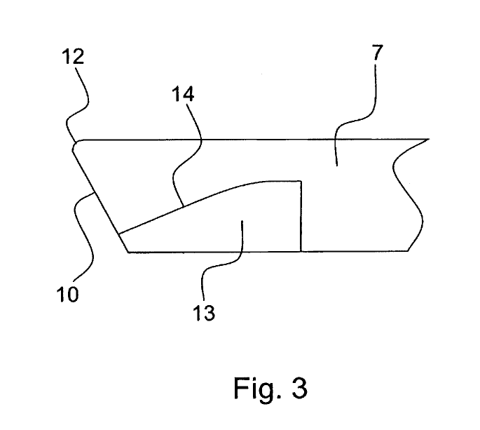

FIG. 3 shows the distal end region of fiber tube 7 in a side view. It can be recognized that fiber tube 7 is designed with a front surface 10 suitable to the front face 11 of medical endoscope 1, wherein the upper corner 12 is rounded and a laterally thicker region 13 is provided with a typical oblique edge 14. Details of this type of construction can be gathered from U.S. Pat. No. 5,718,664 A.

FIG. 3 shows that the distal end region of fiber tube 7 is designed with a very complex geometry. Production in machining process is eliminated due to the use of ceramics. Fiber tube 7 is therefore made of a sintered ceramic. The conventional sintering processes also enable the production of very complex forms in a simple way.

Fiber tube 7 consists of high-strength ceramic, in particular, tungsten carbide. This provides the multiple-tube shaft of endoscope 1 with high strength, in particular high flexural strength. However, there is a risk connected to this, that if the fiber tube is overloaded and breaks, splinters will occur. Cladding tube 2 as the outer tube is made of metal, e.g. suitable high-grade steel. Any splinters that occur are caught by the cladding tube, which cannot splinter. A good patient protection results from this.

System tube 8, which forms the innermost tube, is made in turn of metal, but can also be made of ceramic, in order to increase the strength yet again. It is also possible to form only this tube of ceramic and fiber tube 7 from metal. Cladding tube 2 serves as splinter protection and therefore must always consist of metal.

While there has been shown and described what is considered to be preferred embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

1. A medical endoscope comprising:
a plurality of tubes arranged one inside the other;
wherein an outer tube of the plurality of tubes is made of metal, and at least one inner tube of the plurality of tubes is made of ceramic.

2. The medical endoscope according to claim 1, wherein the ceramic is tungsten carbide.

3. The medical endoscope according to claim 1, wherein the at least one inner tube is configured as a fiber tube inwardly delimiting a fiber chamber filled with optical fibers, and the fiber tube is made of ceramic.

4. The medical endoscope according to claim 3, wherein the fiber tube is designed as formed of a sintered ceramic.
